(19) United States
(12) Patent Application Publication
(10) Pub. No.: US 2024/0124371 A1
(43) Pub. Date: Apr. 18, 2024

(54) NITRIFICATION INHIBITORS AND FORMULATIONS

(71) Applicant: Incitec Fertillsers Operations Pty Ltd, Southbank Victoria (AU)

(72) Inventors: Charles Norman Walker, Southbank Victoria (AU); Roya Khalil, Southbank Victoria (AU); Timothy Hughes, Southbank Victoria (AU)

(21) Appl. No.: 18/257,546

(22) PCT Filed: Dec. 17, 2021
(86) PCT No.: PCT/AU2021/051507

§ 371 Date: Jun. 14, 2023

(30) Foreign Application Priority Data
Dec. 17, 2020 (AU) 2020904709

(51) Int. Cl. C05G 3/90; C05C 3/00; C05G 5/23; C07D 249/04; C07D 249/06; C07D 249/14

(52) U.S. Cl. CPC C05G 3/90; C05C 3/00; C05G 5/23; C07D 249/04; C07D 249/06; C07D 249/14

(57) ABSTRACT

The present invention relates to a nitrification inhibitor formulation for use in combination with anhydrous ammonia, comprising an inhibitor fraction, a solvent fraction and a co-solvent fraction, wherein the inhibitor fraction comprises at least one agriculturally-acceptable inhibitor compound selected from formula 1-6 or a salt thereof, the solvent fraction comprises a polar aprotic solvent, and the co-solvent fraction comprises an ethanolamine compound.

### TECHNICAL FIELD

The present document claims priority from AU2020904709 entitled “Improved Nitrification Inhibitors and Formulations” the contents of which are hereby incorporated in their entirety by reference.

The present invention relates generally to the field of fertilisers, more specifically to liquid nitrogen fertilisers, and in particular to nitrification inhibition of anhydrous ammonia fertilisers.

### BACKGROUND

Many farms are in rural or remote locations. This is particularly evident in countries such as Australia, where farms may be many hundreds of kilometres from the nearest major city. As a result, delivery is time-consuming and expensive, so it is often desirable to utilise agricultural products that are durable and can be stored for extended periods of time in a range of temperatures.

Anhydrous ammonia (AA) is a useful fertiliser that is an excellent source of nitrogen for plants, being approximately 82% nitrogen by weight, making it one of the most mass-efficient nitrogen fertilisers available. AA functions as a fertiliser by dissolving to form an ammonia complex, which is converted by biological oxidation into a nitrite compound, and finally into a nitrate compound, which can be taken up by plants. However, nitrites and nitrates are highly soluble, and so can be rapidly leached from soil within a matter of weeks, particularly with heavy rainfall or other movement of water. Nitrogen as nitrates also represent a substantial environmental concern due to their solubility, toxicity to animals, ability to feed algal blooms, global warming potential and catalysis of ozone degradation. With no form of nitrification inhibitor, AA undergoes biological oxidation into nitrite (and subsequently into nitrates) and, as evidenced by FIG. 1, quickly saturates soil to approximately 100 mg/kg. This is a rapid and potentially shocking release of nitrogen into the soil. It is therefore desirable to be able to control, inhibit or slow down the rate of nitrification of anhydrous ammonia once it is embedded within the soil, enabling an extended release of nitrates at a much lower rate.

Nitrification inhibitors are known in the art, for example Australian Patent 2015227487 for “Nitrification inhibitors and formulations”. Many nitrification inhibitors, such as dimethyl pyrazole compounds, are solid powders under standard conditions. These nitrification inhibitors are also not typically soluble by themselves, requiring modification in order to be useful. For example, the aforementioned prior art patent converts the nitrification inhibitors into alpha hydroxy carboxylic acid additions of various compounds in order to reduce their volatility and/or increase their solubility and thus improve their utility. However, AA is a basic liquid which means that it is typically incompatible with acids and water, as it will exothermically react to form ammonium salts, and therefore the prior art compounds disclosed in the aforementioned Australian patent are not suitable.

FIG. 2 is a process flow diagram for a typical industrial AA applicator. AA is stored in a first pressurised vessel 10, and a nitrification inhibitor (in combination with a solvent) in a second vessel 12. The AA feed line 14 and nitrification inhibitor feed line 16 combine at point 18 before passing through an in-line mixer 20. The mixed liquids pass through distribution line 22 to the applicator boom 24, whereupon the mixture is typically injected, knifed into or otherwise applied to the soil. The skilled person will appreciate that 2 is exemplary only and not to scale. In some forms, the mixture point 18 and in-line mixer may not be present, with separate distribution lines maintained and separate applicator nozzles on the applicator boom 24. This form of the applicator is more complex and so is less desirable.

One prior art solution has been to use tetra-methyl sulfone (‘sulfolane’) as a solvent for nitrification inhibitor compounds, as it is known to be stable with respect to anhydrous ammonia. However, sulfolane has ultimately been found to be a poor long-term solvent for prior art nitrification inhibitor compounds. With reference to FIG. 3, shown are the results of two experiments using sulfolane to dissolve a pyrazole nitrification inhibitor, depicting initial dissolution (left) and subsequent precipitation (right). The solution complex was ultimately found to only be capable of remaining dissolved for a short time before precipitating out of solution or was unable to dissolve at all. As a result of this, the ability of the nitrification inhibitor to stay active for extended lengths of time may be impeded. Similarly, equipment for applying the prior art nitrification inhibitor mixture may become rapidly occluded by precipitate, interrupting the application of the inhibitor to soil.

Owing to the aforementioned rural or remote location of most farms, the nitrification inhibitor is typically stored in tanks (such as vessel 12) for extended periods of time prior to application. This means that there is the risk that prior art nitrification inhibitors using a solvent such as sulfolane may precipitate out of solution in storage, requiring additional expensive equipment to maintain or encourage dissolution. This makes it far more difficult to store the nitrification inhibitor long-term. Additionally, even if the nitrification inhibitor has not substantially precipitated out of solution at the time of use, pumping the inhibitor through the system depicted in FIG. 2 may induce precipitation within the feed line 16, distribution line 22, applicator boom 24 or one of the nozzles, injectors or knifers upon the applicator boom. This leads to clogging, requiring additional maintenance to clean the lines, and—if not noticed immediately—may lead to areas of agricultural land being fertilised with uninhibited AA, leading to rapid loss of fertiliser through nitrification.

It is therefore desirable to provide a means of dissolving a nitrification inhibitor compound, or mixture of compounds, in a stable manner to avoid clogging of equipment. It is further desirable to enable the nitrification inhibitor solution to be stable and compatible with anhydrous ammonia or ammonium solution. It is also desirable to provide a formulation that overcomes or at least ameliorates one or more of the problems exhibited by prior art methods.

These and other advances may be provided by one or more embodiments of the present invention.

### SUMMARY OF THE INVENTION

In a first aspect, the present invention relates to a formulation comprising: anhydrous ammonia; a dissolved inhibitor fraction; a solvent fraction; and a co-solvent fraction.

In another aspect the invention provides a nitrification inhibitor formulation for use in combination with anhydrous ammonia, the formulation comprising an inhibitor fraction, a solvent fraction, and, a co-solvent fraction. In both aspects, the inhibitor fraction comprises at least one agriculturally-acceptable inhibitor compound selected from formula 1-6, or a salt thereof, the solvent fraction comprises a polar aprotic solvent, and the co-solvent fraction comprises an ethanolamine compound.

In an embodiment, the substituents R¹, R² and R³ (if present) are each independently selected from hydrogen, hydroxyl, halo, amino, thio, C₁₋₁₀ alkyl, C₁₋₁₀ alkylamino, C₁₋₁₀ alkylhalo, C₁₋₁₀ alkylthio, C₂₋₁₀ alkenyl, C₂₋₁₀ alkenylamino, C₂₋₁₀ alkenylhalo, C₂₋₁₀ alkenylthio, C₂₋₁₀ alkynyl, C₂₋₁₀ alkynylamino, C₂₋₁₀ alkynylhalo, C₂₋₁₀ alkynylthio, C₁₋₁₀ alkoxy, C₁₋₁₀ alkoxyhalo, and C₁₋₁₀ alkylcarbonylamino, or R¹ and R² for Formula 3 may be joined together to form a 3-10 membered monocyclic or fused bicyclic heteroaryl, with the proviso that the compound is not selected from pyridine citrate, pyridine tartrate, pyridinium tartrate, 1,3,5-triazine-2,4,6-triamine citrate, 1,3,5-triazine-2,4,6-triamine tartrate, ethenylpyridine glycolate, 4-aminopyridinium or 4-dimethylaminopyridine.

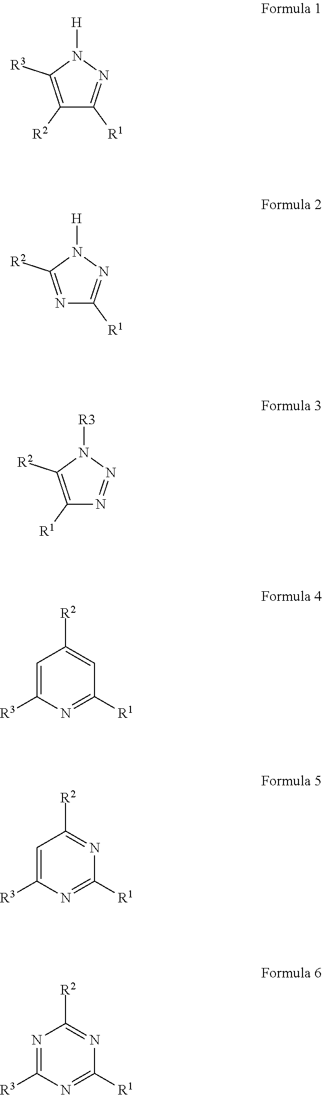

In a further embodiment, the heteroaryl may further be optionally substituted with one or more of the above substituents.

In an embodiment, the inhibitor compound is a pyrazole compound (formula 1). The pyrazole compound can be a dimethyl pyrazole. The dimethyl pyrazole can be 3,4 dimethyl pyrazole. In an embodiment, the inhibitor compound is a triazole compound (formula 2 and 3). The triazole compound can be 1,2,4-triazole or benzotriazole. In an embodiment the inhibitor compound is a pyridine (formula 4). The pyridine can be 2-ethynyl-pyridine or 2-chloro-6-trichloromethyl-pyridine. In an embodiment, the inhibitor compound is a pyrimidine (formula 5). The pyrimidine can be 2-amino-4-chloro-6-methyl-pyrimidine. In an embodiment, the inhibitor compound is a triazine (formula 6). The triazine can be 2,4-diamino-6-trichloromethyl-1,3,5-triazine or 2-amino-4-methyl-6 trichloromethyl-1,3,5-triazine.

The solvent fraction comprises a polar aprotic solvent. The polar aprotic solvent is a solvent that lacks an acidic proton and is polar. Such solvents typically lack hydroxyl and amine groups. These solvents do not serve as proton donors in hydrogen bonding, although they can be proton acceptors. Many solvents, including chlorocarbons and hydrocarbons, are classifiable as aprotic, but polar aprotic solvents are of particular interest for their ability to dissolve salts. Preferably, the polar aprotic solvent is water soluble.

It is thought that any polar aprotic solvent would have the potential to be used in the present formulation, since they are used mainly for the fact they will not react with the anhydrous ammonia when it is mixed and the co-solvent. Further to this, all polar aprotic solvents will be miscible with the ethanolamine due to the similarly polar nature. However, preferably, the aprotic solvent should be one that does not have a high freezing point as that could result in a formulation that freezes during storage or gets too viscous in the cold (commercial application considerations).

The aprotic solvent should be one which is “agriculturally accepted” and therefore is less likely to be phytotoxic. Agriculturally acceptable aprotic solvents are typically already in use in agriculture. Some aprotic solvents that could be available to use, that would likely remain stable in the present formation, include acetone or acetonitrile but these are not common agricultural solvents and will likely be phytotoxic so they are excluded from the discussion here.

The polar aprotic solvent can be selected from one or more of N-Butyl-2-pyrrolidone (NMP), an organosulphur compound including sulfolane and Dimethyl sulfoxide (DMSO) and dihydrolevoglucosenone (cyrene).

The solvent chosen should be miscible with liquid anhydrous ammonia as anhydrous ammonia is typically applied at −77° C., a temperature at which all of the formulations by themselves will freeze so the formulations themselves need to “dissolve” in liquid anhydrous ammonia so they don't freeze and can mix evenly through the anhydrous ammonia. If the chosen solvents are miscible with anhydrous ammonia and dissolve the chosen inhibitor, then the solvents can carry the inhibitor into the anhydrous ammonia as the solvents dissolve in the anhydrous ammonia even if the inhibitor itself alone would not dissolve in the anhydrous ammonia

The preferred solvent is sulfolane, since it is used in anhydrous ammonia production wherein it comes into direct contact with anhydrous ammonia to remove impurities. However other polar aprotic solvents are in scope. Solvents NBP and DMSO, are selected as exemplary since NBP is an emerging replacement for N-Methyl-2-pyrrolidone (NMP) which is being phased out due to recent reports of it as a potential human reproductive hazard. DMSO is a very strong solvent and has also seen use at improving stability of “tank mixes” which is where a farmer mixes products together to allow one application of multiple products.

The co-solvent fraction is an ethanolamine. Co-solvents are substances added to a primary solvent fraction in small amounts to increase the solubility of a poorly-soluble compound. The co-solvent added to the solvent fraction can be to assist the solvent fraction in keeping the nitrification inhibitor in solution.

In an embodiment, the co-solvent is a pH balancer. The formulation will come into direct contact with anhydrous ammonia which is extremely basic. Without wishing to be bound by theory it is thought that by making the formulation basic the possibility of reaction with the anhydrous ammonia may be lessened. A reaction with anhydrous ammonia would be very dangerous (hence the formulation is water and acid free). The basic nature of TEA comes from the amine and alcohol groups. These groups are also thought to assist in improving the solubility of the inhibitor compounds as they all possess amine groups. As with e.g. sulfolane, triethanolamine (TEA) is also used in ammonia production and is itself made from ammonia so the chance of reaction with anhydrous ammonia upon contact is very low.

In an embodiment, the ethanolamine compound is diethanolamine or triethanolamine. In an embodiment, the co-solvent fraction comprises both diethanolamine and triethanolamine. Other ethanolamines such as monoethanolamine, N-Isopropylethanolamine, 2-Dimethylaminoethanol, N-Butyldiethanolamine and similar are also in scope.

In an embodiment, the solvent fraction is at least 45 wt. % thereof. In an embodiment, the co-solvent fraction is greater than 25 wt. % thereof.

In an embodiment, the inhibitor fraction is a mixture of two or more compounds, each being selected from the group consisting of formula 1-6 or a salt thereof. In an embodiment, at least two of the two or more compounds are the same selection of Formula 1-6, but have different substitutions for at least one of R¹, R² or R³ (if present).

These and other embodiments may become apparent to the skilled person through the disclosure contained herein, and are considered to fall within the scope of the present invention.

The invention also provides a method of dissolving an inhibitor fraction as described herein into solution in the presence of anhydrous ammonia. Also provided is an anhydrous ammonia fertilizer solution comprising the formulation described herein or prepared by any one of the methods described herein.

### DEFINITION OF TERMS

The term “agriculturally acceptable” includes solvents or additives that are suitable for agricultural formulations. For example “agriculturally acceptable excipient” includes but is not limited to water, alcohols such as butanol or glycol as well as their ethers and esters, ketones such as acetone, methyl ethyl ketone, sulfones such as dialkyl sulfones, polymethylene cyclic sulfones, petroleum fractions, mineral or vegetable oils, aliphatic chlorinated hydrocarbons, water-soluble or strongly polar solvents, liquefied gases such as halogenated hydrocarbons as well as butane, propane, nitrogen and carbon dioxide, carboxymethyl cellulose, ethylene glycol, propylene glycol, kaolin, bentonite, dolomite, calcium carbonate, talc, powder magnesia, Fuller's earth, gypsum, diatomaceous earth and China clay.

### DESCRIPTION OF FIGURES

Embodiments of the present invention will now be described in relation to figures which are exemplary only and which are not limiting and wherein:

FIG. 1 is a graph depicting concentration of nitrates in soil from uninhibited anhydrous ammonia;

FIG. 2 depicts a typical A process flow diagram for an AA and nitrification inhibitor applicator;

FIG. 3 depicts a prior art nitrification inhibitor precipitating out of solution;

FIG. 4 is a graph depicting levels of nitrogen in soil as ammonium nitrogen, for uninhibited AA, AA with a prior art nitrification inhibitor and AA with an embodiment of the present invention;

FIGS. 5A-5D depict solubilities of various embodiments of the formulation of the present invention; and

FIG. 6 is a graph depicting stability of a dimethyl pyrazole compound in various scenarios.

FIG. 7 shows Table 1 which provides sample preparation details and Table 2 which provides preparation procedures.

FIG. 8 is Table 2 which provides visual observations of samples stored at ambient temps and Table 4 provides visual observations of samples stored at low temps.

FIG. 9 is Table showing formulations made and described in Examples 3 to 5.

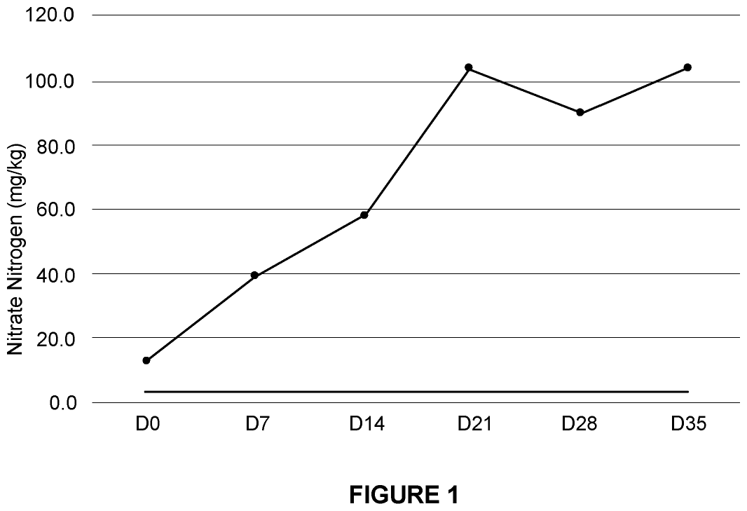

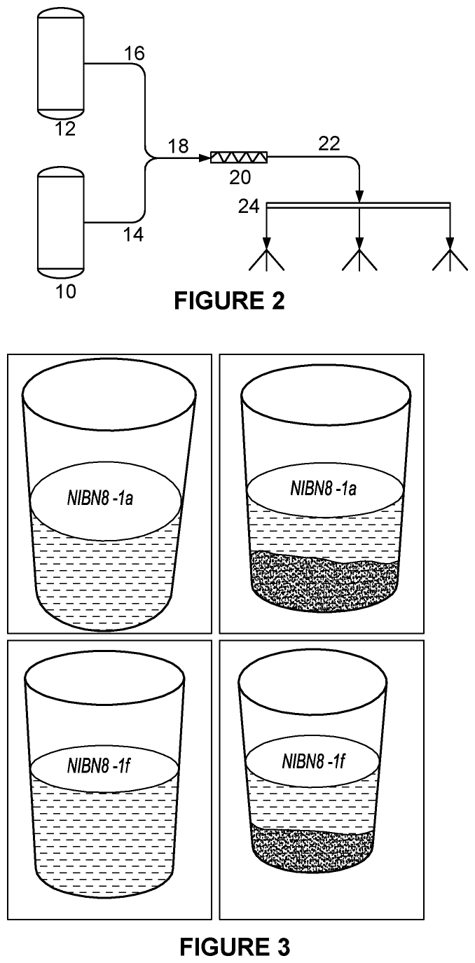

### DETAILED DESCRIPTION OF PREFERRED EMBODIMENTS

In a first aspect there is provided an improved nitrification inhibitor formulation for use in combination with anhydrous ammonia (AA), comprising an inhibitor compound, a polar aprotic solvent and an ethanolamine compound. It is considered that the addition of a polar aprotic solvent and an ethanolamine compound may aid agriculturally-acceptable inhibitor compounds to stay in solution for extended periods of time whilst remaining compatible with anhydrous ammonia. This may enable the nitrification inhibitor formulation to be applied with either no risk or at least a substantially reduced risk of feed lines, nozzles and/or tubing within an applicator becoming clogged with precipitated inhibitor compound.

thereby inhibiting the rate of nitrification of the anhydrous ammonia for longer. In an embodiment, the formulation comprises an inhibitor fraction that contains the inhibitor compound, a solvent fraction that contains the polar aprotic solvent, and a co-solvent fraction that contains the ethanolamine compound.

In an embodiment, the nitrification inhibitor compound may be selected from the group consisting of Formulae 1 to 6, as laid out below:

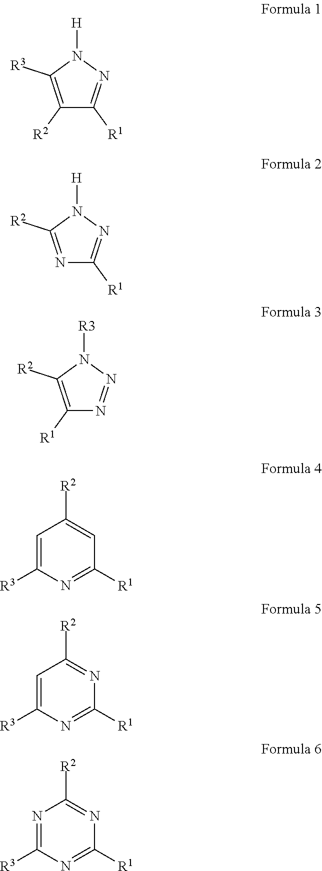

### Wherein:

Formula 1 is a 3,4,5-substituted pyrazole;
Formula 2 is a 3,5-substituted 1,2,4-triazole;
Formula 3 is a 4,5-substituted 1,2,3-triazole;
Formula 4 is a 2,4,6-substituted pyridine;
Formula 5 is a 2,4,6-substituted pyrimidine; and
Formula 6 is a 2,4,6-substituted 1,3,5-triazine.

In an embodiment, R1, R2 and R3 (if present) are each independently selected from hydrogen, hydroxyl, halo, amino, thio, C₁₋₁₀ alkyl, C₁₋₁₀ alkylamino, C₁₋₁₀ alkylhalo, C₁₋₁₀ alkylthio, C₂₋₁₀ alkenyl, C₂₋₁₀ alkenylamino, C₂₋₁₀ alkenylhalo, C₂₋₁₀ alkenylthio, C₂₋₁₀ alkynyl, C₂₋₁₀ alkynylamino, C₂₋₁₀ alkynylhalo, C₂₋₁₀ alkynylthio, C₁₋₁₀ alkoxy, C₁₋₁₀ alkoxyhalo, and C₁₋₁₀ alkylcarbonylamino.

In an embodiment wherein the nitrification inhibitor compound is or comprises Formula 3, R¹, R² and/or R³ may be joined together to form a 3-10 membered monocyclic or fused bicyclic heteroaryl. In a further embodiment, the heteroaryl may further be optionally substituted with one or more of the above substituents. In an embodiment, the compound of formula 3 may be selected from 4-butyl-1H-1,2,3-triazole-1-butanoic acid ethyl ester; 2-[3-[4,5-di(hydroxymethyl)-1H-1,2,3-triazole]propyl]-isoindoline-1,3-dione; 2-[3-[4,5-(methyl ethanoate)-1H-1,2,3-triazole]propyl]-isoindoline-1,3-dione; ethyl 4,5-bis(hydroxymethyl)-1H-1,2,3-triazole-1-butyrate; ethyl 4,5-bis(methyl ethanoate)-1H-1,2,3-triazole-1-butyrate; ethyl 4,5-bis(methyl ethanoate)-1H-1,2,3-triazole-1-acetate; 1-butyl-4-propyl-1H-1,2,3-triazole; 1-(2-methoxyethyl)-4-butyl-1H-1,2,3-triazole; 4-propyl-1H-1,2,3-triazole-1-ethanol; and 1-(3-butyn-1-yl)-4-propyl-1H-1,2,3-triazole; or agriculturally acceptable salts thereof.

In another embodiment, the inhibitor compound may be selected from the group consisting of 3-methyl pyrazole, 3,4-dimethyl pyrazole, 1,2,4-triazole, benzotriazole, 2-ethynyl-pyridine, 2,4-diamino-6-trichloromethyl-1,3,5-triazine, 2-amino-4-methyl-6-trichloromethyl-1,3,5-triazine, 2-chloro-6-trichloromethyl-pyridine and 2-amino-4-chloro-6-methyl-pyrimidine or agriculturally acceptable salts thereof.

In a further embodiment, the nitrification inhibitor compound is not selected from pyridine citrate, pryridine tartrate, pyridinium tartrate, 1,3,5-triazine-2,4,6-triamine citrate, 1,3,5-triazine-2,4,6-triamine tartrate, or ethenylpyridine glycolate.

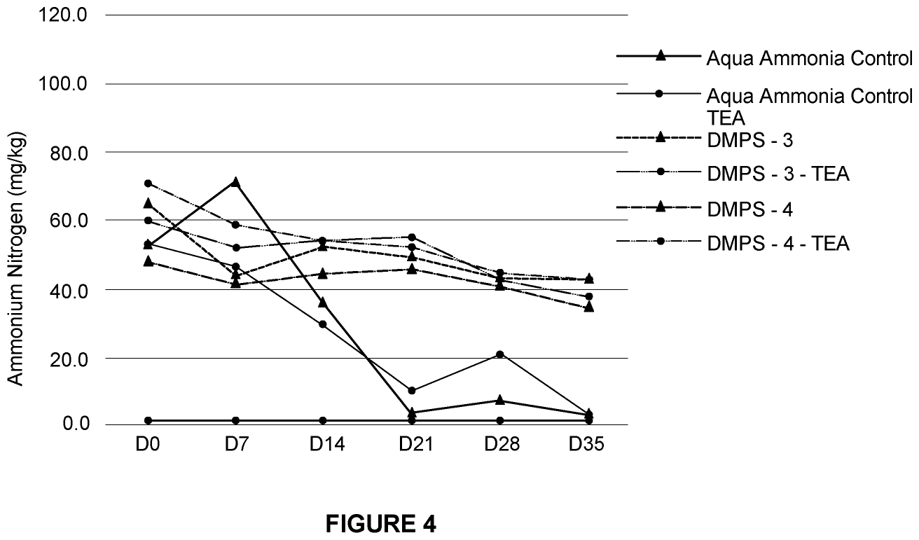

FIG. 4 depicts an analysis of the ability for ammonium nitrogen to be stabilised within soil by various nitrification inhibitors. As shown therein, a formulation comprising an inhibitor fraction and a solvent fraction (EG: DMP Sulfolane-3 (DMPS-3), DMP Sulfolane-4 (DMPS-4)) are equalled or outperformed by embodiments of the present invention, being a formulation comprising an inhibitor fraction, a solvent fraction and a co-solvent fraction (EG: DMP Sulfolane-3-TEA (DMPS-3-TEA), DMP Sulfolane-4-TEA (DMPS-4-TEA)). As such, and without limiting the scope of the invention through theory, it is believed that embodiments of the present invention utilizing both a solvent and a co-solvent enable the inhibitor compounds within the inhibitor fraction to remain in solution without limiting the effectiveness of the inhibitor fraction.

In at least one embodiment of the present invention, the inhibitor compound is a pyrazole compound, in particular a compound that has the formula depicted in Formula 1. In a further embodiment, the pyrazole compound is a dimethyl pyrazole (DMP). In a further embodiment, the dimethyl pyrazole is one of 3,4 dimethyl pyrazole or 3,5 dimethyl pyrazole. In embodiments wherein the inhibitor compound is a dimethyl pyrazole, the inhibitor compound may be present as a dimethyl pyrazole salt. The dimethyl pyrazole salt may be a dimethyl pyrazole phosphate salt.

In an embodiment of the present invention, the polar aprotic solvent present in the solvent fraction may be an organosulphur compound. In a further embodiment, the organosulphur compound may be tetramethylene sulphone (sulfolane). In such an embodiment, the solvent fraction may comprise at least 45 wt. % of the formulation.

In an embodiment of the present invention, the ethanolamine compound may be triethanolamine (‘TEA’). The ethanolamine compound may be diethanolamine (DEA). In an embodiment, the co-solvent fraction may be a combination of TEA and DEA. Without limiting the scope of the invention, TEA and DEA are considered to be a suitable co-solvent due to their ability as a pH balancer, which may assist an embodiment of the formulation of the present invention in remaining stable in combination with AA.

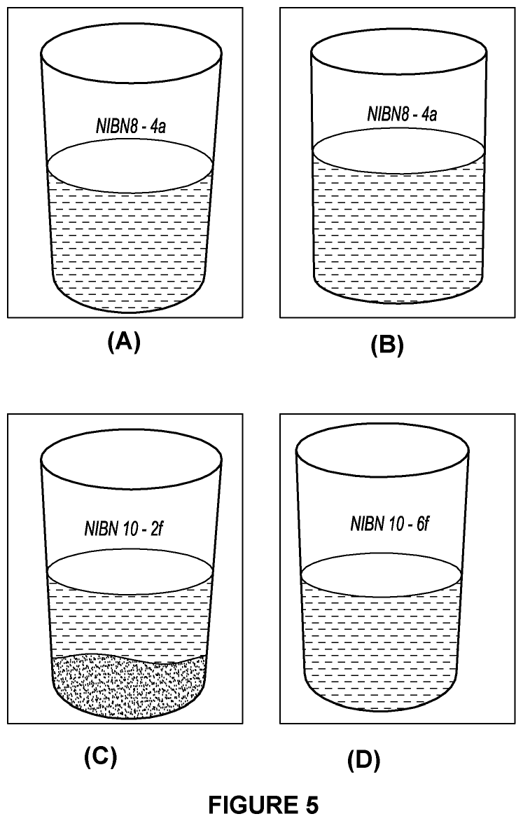

Referring now to FIG. 5A-D, there are depicted four solutions. FIGS. 5A and 5B depict an embodiment of the formulation, wherein the co-solvent fraction comprises 50 wt. %, upon preparation (5A) and after fourteen days (5B). No precipitation is observed. Upon reducing the co-solvent fraction to 25 wt. %, however, precipitation is observed within 14 days, as shown in FIG. 5C. Increasing the co-solvent fraction to 30 wt. %, as in FIG. 5, resulted in no precipitation being observed after 14 days. Therefore, in an embodiment, the co-solvent fraction may be greater than 25 wt. % of the formulation composition.

As ethanolamine compounds (in particular DEA and TEA) are viscous, it is considered that optimising the formulation composition requires lowering the viscosity of the formulation as much as is practical. As a result of experimentation, it is further theorised that an optimum weight fraction for the co-solvent fraction lies between 25 wt. % and 35 wt. %. In a further embodiment of the present invention, the co-solvent fraction may comprise between 27.0 wt. % and 30.0 wt. % of the formulation composition. In at least one specific embodiment, the co-solvent fraction may comprise 27.4 wt. % of the formulation composition.

In an embodiment, the ethanolamine can be used in a higher percentage to give the solubility and then “thinned out” with the polar aprotic solvent to lower the viscosity and reduce the freezing point. However, the end result is still a optimum weight fraction for the co-solvent fraction lies between 25 wt. % and 35 wt. %.

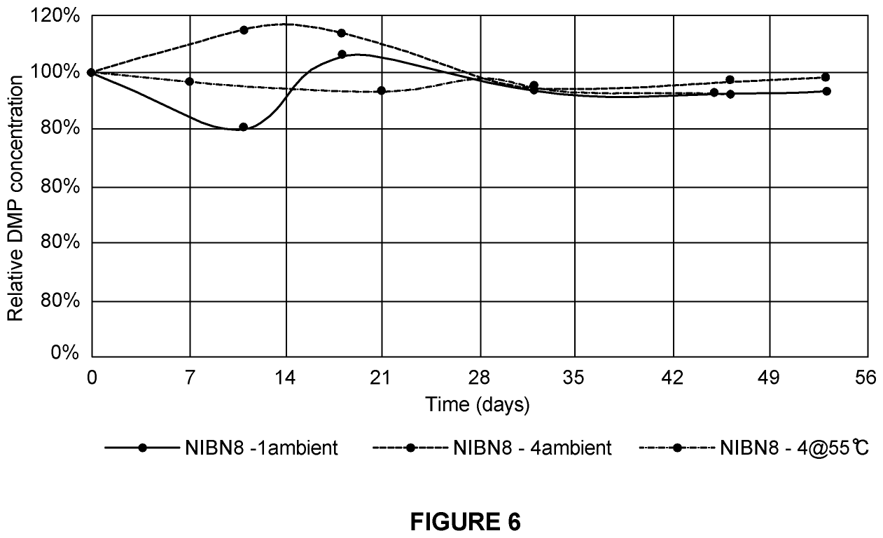

FIG. 6 depicts a chemical stability chart of a prior art formulation comprising dimethyl pyrazole and sulfolane (DMP-S) (blue) as well as an embodiment of the present invention comprising a dimethyl pyrazole compound, sulfolane and a co-solvent fraction comprising at least TEA (orange: ambient temperature; grey: 55° C.). DMP-S is known to be stable, and this is confirmed by the referenced figure. However, the figure also establishes that dimethyl pyrazole compounds are stable in combination with both sulfolane and TEA, both at ambient temperature and at elevated temperatures.

Therefore, without limiting the scope of the invention through theory, it is considered that at least one embodiment of the present invention, comprising a dimethyl pyrazole compound, sulfolane and at least TEA, will provide similar nitrification inhibition capabilities to dimethyl pyrazoles either alone or in combination with just sulfolane, while providing improved solubility in AA and/or basic conditions. As such, a formulation of the present invention may provide nitrification inhibition over an extended period of time, thereby improving soil stability, and reducing or ameliorating some of the disadvantageous effects of nitrates.

The description herein is directed to use of the formulation with anhydrous ammonia (which is a liquid when applied to soil as it is under pressure at −77° C.). However, there is nothing preventing the application of the formulation to granules. The key here is that we chose classes of solvents that will not react with anhydrous ammonia and will be miscible with anhydrous ammonia. We need them to be miscible as anhydrous ammonia is typically applied at −77° C., a temperature at which all of the formulations by themselves will freeze so the formulations themselves need to “dissolve” in anhydrous ammonia so they don't freeze and mix evenly through the anhydrous ammonia. If the chosen solvents are miscible with anhydrous ammonia and dissolve the chosen inhibitor well, then the solvents can carry the inhibitor into the anhydrous ammonia as the solvents dissolve in the anhydrous ammonia even if the inhibitor itself alone would not dissolve in the anhydrous ammonia.

### EXAMPLES

The following examples illustrate embodiments of the invention and other embodiments and are intended to be exemplary only and not limiting.

### Example 1

### Physical Stability Testing

NIBN8-1 comparative using DMP, sulfolane
NIBN8-2 comparative using DMP, sulfolane
NIBN8-3 comparative using DMP, sulfolane
NIBN8-4 embodiment using DMP, sulfolane, TEA
NIBN8-5 embodiment using DMP, sulfolane, TEA

Formulations made without heating were prepared according to the method: The solvent mixture was stirred for 3-5 mins at 500 rpm in a 600 mL glass beaker, whilst the required amount of DMP was added. After which the stirring was increased to 750 rpm and the mixture stirred for a further 10 minutes.

Formulations made with heat were prepared according to the method below: The solvent was heated for upwards of 5 mins on ‘high’ whilst stirring at 500 rpm to a temperature of >70 ° C. in a 600 mL glass beaker. The required amount of DMP was added to the hot solvent whilst stirring after which the stirring was increased to 750 rpm and the heat setting dropped to ‘3.5’ to maintain the temperature between 70-90 ° C. and stirred for a further 10 minutes.

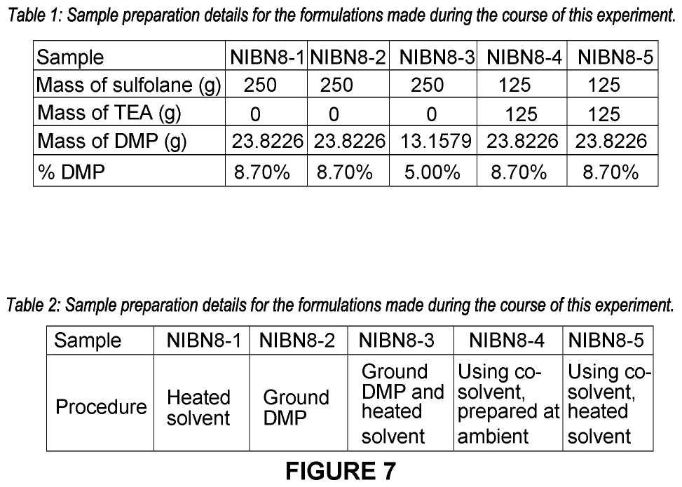

Formulations made with ground DMP were made the same as the “without heating” formulations except the DMP was ground using a coffee grinder on setting “fine”. Stirring for all was provided by an overhead stirrer. Tables 1 and 2 of FIG. 7 show the details of each formulation tested and the sample preparation methods. Formulation NIBN8-2 did not pass sample preparation, so it not discussed further.

Multiple of the formulations made according to the same methods were labelled a, b, c, d, e, f and so on. For example, NIBN8-4a is the same formulation as NIBN8-4f.

The samples were stored in the R&D lab and fridge for 2 weeks. Observations and images were taken at intervals across the 2 weeks. Temperature and relative humidity conditions at ambient and within the fridge were monitored with Hobo UX100-003 data loggers. During the experiment the average temperature of the ambient lab was 19.8° C. and that of the fridge was 4.3° C., as logged by the temperature and humidity data loggers in close proximity to the samples.

After preparation, the samples were allowed to stand (for at least 20 minutes) and cool to room temperature (if heat was applied). This was necessary as, due to the viscosity of some formulations, bubbles had formed throughout causing identification of remaining solids difficult, and the heat of the heated solutions could have compromised the plastic. Clear solutions were formed for all formulations except NIBN8-2.

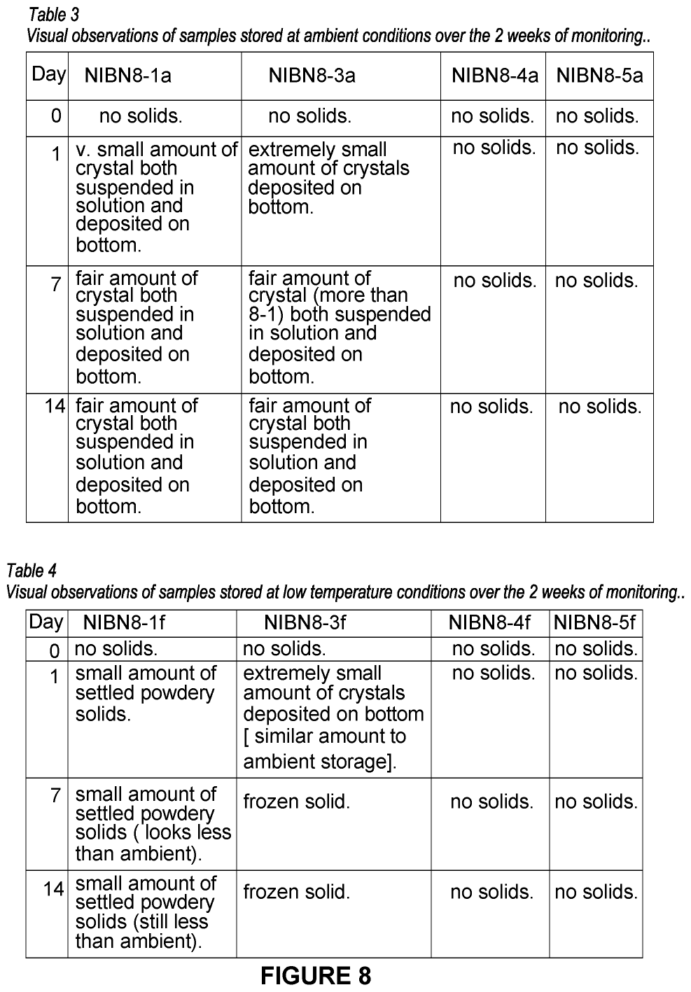

All successful formulations remained stable in solution. The Tables of FIG. 8 summarize the physical stability observations over the 2 weeks of monitoring. As NIBN8-2 did not pass sample preparation, it was not included in the physical stability monitoring.

Conclusion: The use of TEA as a co-solvent was seen as an effective method for improving the stability of DMP in the formulation, showing no precipitation in ambient or low temperatures over 2 weeks. The DMP-Sulfolane (DMP-S) formulations without TEA showed precipitation after a day of storage, indicating heating of the solvent before and during mixing as well as the use of ground DMP were not effective, alone or combined, in making a stable DMP-S formulation.

### Example 2

### Optimisation of the Solvent: Co-Solvent Ration in the Formulation

NIBN10-1 embodiment using DMP, sulfolane (S), TEA; S:TEA=60:40
NIBN10-2 embodiment using DMP, sulfolane, (S), TEA; S:TEA=75:25
NIBN10-3 embodiment using DMP, sulfolane, (S), TEA; S:TEA=80:20
NIBN10-6 embodiment using DMP, sulfolane, (S), TEA; S:TEA=70:30

The samples prepared for this experiment represent a step-wise decrease in TEA percentage from the 50:50 ratio of sulfolane to TEA. All formulations were made using the overhead stirrer, with the TEA and sulfolane mixed for 5 minutes at 500 rpm before addition of DMP, after which the mixture was stirred for 10 minutes at 900 rpm

Formulations were made in order from NIBN10-1 to NIBN10-6. Formulation 10-1 performed as intended, with the DMP dissolving completely after the 10 minutes of stirring. However, NIBN10-2 and NIBN10-3 both had remaining undissolved DMP after stirring indicating the amount of TEA was not sufficient. As the remaining undissolved DMP in NIBN10-2 was minimal, the inclusion of a further 5% of TEA was sufficient to cause complete dissolution and formation of a clear solution. Formulation NIBN10-6 was split into two containers: one stored at ambient conditions and the other stored in the fridge. No precipitation was seen when formulation NIBN10-6 was stored at ambient and low temperature conditions for two weeks. This shows that the 70:30 sulfolane to TEA ratio is sufficient to initially solubilise and keep DMP in solution at typical storage conditions.

The minimum amount of TEA possible in the DMP-Sulfolane/TEA formulation was determined to be 27.4%, or a 70:30 sulfolane:TEA ratio. This formulation showed complete dissolution of the required amount of DMP as well as sustained physical stability over two weeks storage at ambient and low temperature conditions.

### Example 3

### Demonstration of the Use of Various Polar Aprotic Solvents

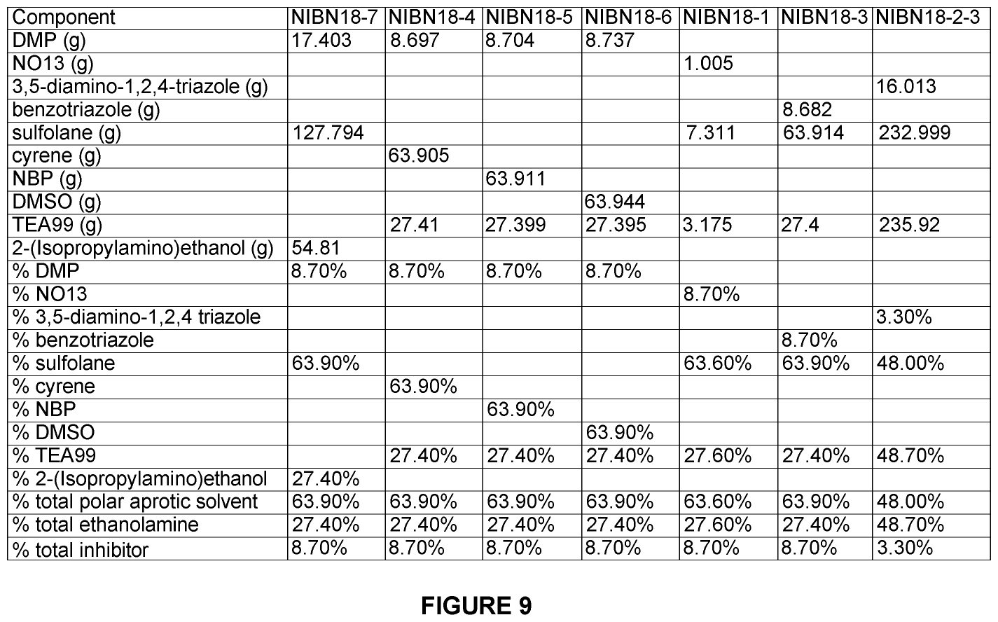

The Table of FIG. 9 shows the formulations used for Examples 3 to 5.
NIBN18-4 embodiment using DMP, cyrene, TEA
NIBN18-5 embodiment using DMP, NBP, TEA
NIBN18-6 embodiment using DMP, DMSO, TEA

The solvent mixture was stirred for 3-5 mins at 650 rpm in a 250 mL plastic jar, whilst the required amount of inhibitor was added. After which the mixture was stirred for a further 10 minutes or until clear, whichever was greatest.

The samples were stored in the R&D lab bench, oven and fridge for 2 weeks. Observations and images were taken at intervals across the 2 weeks. Temperature and relative humidity conditions at ambient and within the fridge were monitored with Hobo UX100-003 data loggers. During the experiment the average temperature of the ambient lab was 19.8° C., that of the oven was 54.0° C. and that of the fridge was 4.3° C., as logged by the temperature and humidity data loggers in close proximity to the samples.

All formulations remained stable in solution. It therefore concluded that polar aprotic solvents other than sulfolane, are able to be subject to the present formulation.

### Example 4

### Demonstration of the Use of Various Ethanolamines

NIBN18-7 embodiment using DMP, sulfolane, N-Isopropylethanolamine (IPEA)

The samples were stored in the R&D lab bench, oven and fridge for 2 weeks. Observations and images were taken at intervals across the 2 weeks. Temperature and relative humidity conditions at ambient and within the fridge were monitored with Hobo UX100-003 data loggers. During the experiment the average temperature of the ambient lab was 19.8° C., that of the oven was 54.0° C. and that of the fridge was 4.3° C., as logged by the temperature and humidity data loggers in close proximity to the samples.

All formulations remained stable in solution. It therefore concluded that ethanolamines other than TEA are able to be subject to the present formulation.

### Example 5

### Demonstration of the Use of Various NI

NIBN18-1 embodiment using 1-butyl-4-propyl-1H-1,2,3-triazole, sulfolane (formula 3), TEA
NIBN18-3 embodiment 3,5-diamino-1,2,4-triazole (formula 2), sulfolane, TEA
NIBN18-2-3 embodiment using benzotriazole (formula 3), sulfolane, TEA

The solvent mixture was stirred for 3-5 mins at 650 rpm in a 250 mL plastic jar, whilst the required amount of inhibitor was added. After which the mixture was stirred for a further 10 minutes or until clear, whichever was greatest. In the case where a smaller scale formulation was made, a 25 mL glass beaker was used in place of the 250 mL plastic jar.

The samples were stored in the R&D lab bench, oven and fridge for 2 weeks. Observations and images were taken at intervals across the 2 weeks. Temperature and relative humidity conditions at ambient and within the fridge were monitored with Hobo UX100-003 data loggers. During the experiment the average temperature of the ambient lab was 19.8° C., that of the oven was 54.0° C. and that of the fridge was 4.3° C., as logged by the temperature and humidity data loggers in close proximity to the samples.

All formulations remained stable in solution. It therefore concluded that many nitrification inhibitors are able to be subject to the present formulation.

While the invention has been described with reference to preferred embodiments above, it will be appreciated by those skilled in the art that it is not limited to those embodiments, but may be embodied in many other forms, variations and modifications other than those specifically described. The invention includes all such variation and modifications. The invention also includes all of the steps, features, components and/or devices referred to or indicated in the specification, individually or collectively and any and all combinations or any two or more of the steps or features.

In this specification, unless the context clearly indicates otherwise, the word “comprising” is not intended to have the exclusive meaning of the word such as “consisting only of”, but rather has the non-exclusive meaning, in the sense of “including at least”. The same applies, with corresponding grammatical changes, to other forms of the word such as “comprise”, etc.

In this specification and unless otherwise specified, any and all chemistry terms used herein or visual depictions of chemical formulae should be interpreted and understood in accordance with the standard chemical nomenclature and terminology as established, at the priority date of this specification, by the International Union of Pure and Applied Chemistry (IUPAC), or otherwise in accordance with standard industry usage of the term.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Any promises made in the present document should be understood to relate to some embodiments of the invention, and are not intended to be promises made about the invention in all embodiments. Where there are promises that are deemed to apply to all embodiments of the invention, the applicant/patentee reserves the right to later delete them from the description and they do not rely on these promises for the acceptance or subsequent grant of a patent in any country.

The description as it relates to any one of the aspects described applies equally to all other aspects unless the context makes clear otherwise.

1. A formulation comprising:
anhydrous ammonia;
a dissolved inhibitor fraction;
a solvent fraction; and
a co-solvent fraction;
wherein the inhibitor fraction comprises at least one agriculturally-acceptable nitrification inhibitor compound selected from Formula 1-6, or a salt thereof: 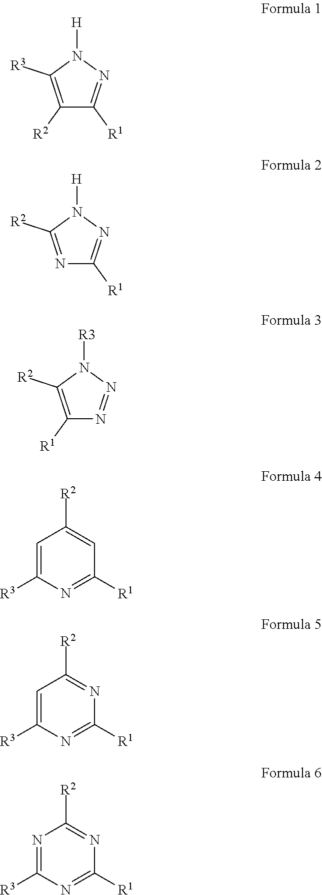
the substituents R¹, R² and R³ (if present) are each independently selected from hydrogen, hydroxyl, halo, amino, thio, C₁₋₁₀ alkyl, C₁₋₁₀ alkylamino, C₁₋₁₀ alkylhalo, C₁₋₁₀ alkylthio, C₂₋₁₀ alkenyl, C₂₋₁₀ alkenylamino, C₂₋₁₀ alkenylhalo, C₂₋₁₀ alkenylthio, C₂₋₁₀ alkynyl, C₂₋₁₀ alkynylamino, C₂₋₁₀ alkynylhalo, C₂₋₁₀ alkynylthio, C₁₋₁₀ alkoxy, C₁₋₁₀ alkoxyhalo, and C₁₋₁₀ alkylcarbonylamino, or R¹, R² and/or R³ for Formula 3 are joined together to form a 3-10 membered monocyclic or fused bicyclic heteroaryl;
with the proviso that the compound is not selected from pyridine citrate, pyridine tartrate, pyridinium tartrate, I,3,5-triazine-2,4,6-triamine citrate, 1,3,5-triazine-2,4,6-triamine tartrate, ethenylpyridine glycolate, 4-aminopyridinium or 4-dimethylaminopyridine;
the solvent fraction comprises a polar aprotic solvent; and
the co-solvent fraction comprises an ethanolamine compound.

2. A nitrification inhibitor formulation for use in combination with anhydrous ammonia, the formulation comprising:
an inhibitor fraction;
a solvent fraction; and
a co-solvent fraction;
wherein the inhibitor fraction comprises at least one agriculturally-acceptable nitrification inhibitor compound selected from Formula 1-6, or a salt thereof: 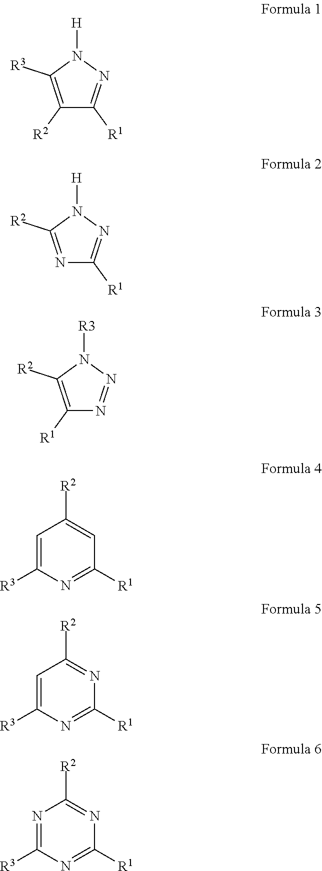
the substituents R¹, R² and R³ (if present) are each independently selected from hydrogen, hydroxyl, halo, amino, thio, C₁₋₁₀ alkyl, C₁₋₁₀ alkylamino, C₁₋₁₀ alkylhalo, C₁₋₁₀ alkylthio, C₂₋₁₀ alkenyl, C₂₋₁₀ alkenylamino, C₂₋₁₀ alkenylhalo, C₂₋₁₀ alkenylthio, C₂₋₁₀ alkynyl, C₂₋₁₀ alkynylamino, C₂₋₁₀ alkynylhalo, C₂₋₁₀ alkynylthio, C₁₋₁₀ alkoxy, C₁₋₁₀ alkoxyhalo, and C₁₋₁₀ alkylcarbonylamino, or R¹, R² and/or R³ for Formula 3 are joined together to form a 3-10 membered monocyclic or fused bicyclic heteroaryl;
with the proviso that the compound is not selected from pyridine citrate, pyridine tartrate, pyridinium tartrate, I,3,5-triazine-2,4,6-triamine citrate, 1,3,5-triazine-2,4,6-triamine tartrate, ethenylpyridine glycolate, 4-aminopyridinium or 4-dimethylaminopyridine;
the solvent fraction comprises; and
the co-solvent fraction comprises an ethanolamine compound.

3. The formulation of claim 1 or 2 wherein the inhibitor compound comprises a compound of Formula 3, or a salt thereof; and
at least one of the members of the hereteroaryl is a substituent selected from hydrogen, hydroxyl, halo, amino, thio, C₁₋₁₀ alkyl, C₁₋₁₀ alkylamino, C₁₋₁₀ alkylhalo, C₁₋₁₀ alkylthio, C₂₋₁₀ alkenyl, C₂₋₁₀ alkenylamino, C₂₋₁₀ alkenylhalo, C₂₋₁₀ alkenylthio, C₂₋₁₀ alkynyl, C₂₋₁₀ alkynylamino, C₂₋₁₀ alkynylhalo, C₂₋₁₀ alkynylthio, C₁₋₁₀ alkoxy, C₁₋₁₀ alkoxyhalo, and C₁₋₁₀ alkylcarbonylamino.

4. The formulation of claim 1 or 2 wherein the inhibitor compound is a pyrazole compound.

5. The formulation of claim 4 wherein the pyrazole compound is a dimethyl pyrazole.

6. The formulation of claim 5 wherein the dimethyl pyrazole is 3,4 dimethyl pyrazole.

7. The formulation of any one of claims 1 to 6, wherein the polar aprotic solvent is an organosulphur compound.

8. The formulation of claim 7 wherein the organosulphur compound is sulfolane.

9. The formulation of any one of claims 1 to 8, wherein the ethanolamine compound is diethanolamine or triethanolamine.

10. The formulation of claim 9, wherein the co-solvent fraction comprises both diethanolamine and triethanolamine.

11. The formulation of any one of claims 1 to 10, wherein the solvent fraction is at least 45 wt. % of the formulation.

12. The formulation of any one of claims 1 to 11, wherein the co-solvent fraction is in the range of from about 25 wt. % to about 30.0 wt % of the formulation.

13. The formulation of claim 11 or claim 122, wherein the inhibitor fraction is a mixture of two or more compounds, each being selected from the group consisting of formula 1-6 or a salt thereof.

14. The formulation of claim 133, wherein at least two of the two or more compounds are the same selection of Formula 1-6, but have different substitutions for at least one of R¹, R² or R³ (if present).

15. The formulation of any one of claims 1 to 14 being a liquid formulation.

16. A method of dissolving an inhibitor fraction into solution in the presence of anhydrous ammonia, the inhibitor fraction comprising at least one agriculturally-acceptable nitrification inhibitor compound selected from Formula 1-6, or a salt thereof: 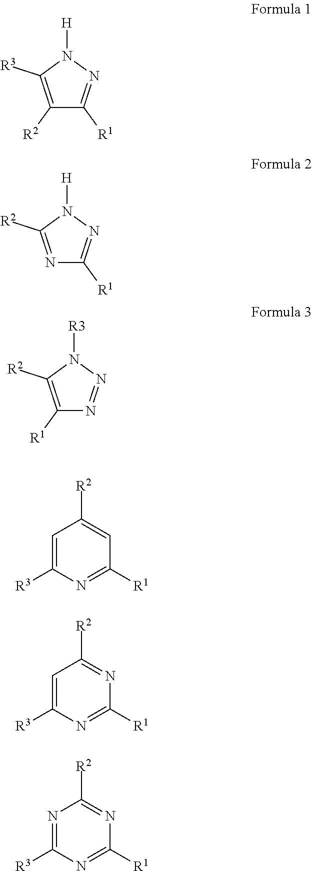
the substituents R¹, R² and R³ (if present) are each independently selected from hydrogen, hydroxyl, halo, amino, thio, C₁₋₁₀ alkyl, C₁₋₁₀ alkylamino, C₁₋₁₀ alkylhalo, C₁₋₁₀ alkylthio, C₂₋₁₀ alkenyl, C₂₋₁₀ alkenylamino, C₂₋₁₀ alkenylhalo, C₂₋₁₀ alkenylthio, C₂₋₁₀ alkynyl, C₂₋₁₀ alkynylamino, C₂₋₁₀ alkynylhalo, C₂₋₁₀ alkynylthio, C₁₋₁₀ alkoxy, C₁₋₁₀ alkoxyhalo, and C₁₋₁₀ alkylcarbonylamino, or R¹, R² and/or R³ for Formula 3 are joined together to form a 3-10 membered monocyclic or fused bicyclic heteroaryl;
with the proviso that the compound is not selected from pyridine citrate, pyridine tartrate, pyridinium tartrate, I,3,5-triazine-2,4,6-triamine citrate, 1,3,5-triazine-2,4,6-triamine tartrate, ethenylpyridine glycolate, 4-aminopyridinium or 4-dimethylaminopyridine;
the method comprising the steps of:
dissolving the inhibitor fraction into a solvent fraction comprising a polar aprotic solvent; and a co-solvent fraction comprising an ethanolamine compound.

17. The method of claim 16, wherein the solvent fraction and co-solvent fraction are mixed to form a mixture, then the inhibitor fraction is added to the mixture.

18. The method of claim 16 or 17, wherein the solvent fraction is at least 45 wt. % of the formulation.

19. The method of any one of claims 16 to 18, wherein the co-solvent fraction is in the range of from about 25 wt. % to about 30.0 wt. % of the formulation.

20. An anhydrous ammonia fertilizer solution comprising the formulation of any one of claims 2 to 15 or prepared by any one of the methods of claims 16 to 19.
